(19) United States
(12) Patent Application Publication
(10) Pub. No.: US 2012/0101336 A1
(43) Pub. Date: Apr. 26, 2012

(54) ENDOSCOPIC SYSTEM WITH FLUID CLEANING

(76) Inventors: Yoav Hirsch, Modiin (IL); Ilan Carmel, Tel Mond (IL); Tal Duvdevany, Tel Aviv (IL); Izhak Fabian, Kfar Truman (IL); Tal Gordon, Hod HaSharon (IL)

(21) Appl. No.: 13/322,595

(22) PCT Filed: May 26, 2010
(86) PCT No.: PCT/US2010/036093

§ 371 Date: Jan. 11, 2012

Related U.S. Application Data
(60) Provisional application No. 61/181,356, filed on May 27, 2009.

(51) Int. Cl. A61B 1/12

(52) U.S. Cl. 600/156

(57) ABSTRACT

An endoscopic system for use with an endoscope, including an irrigation tube that provides a flow of irrigation fluid for cleaning a body lumen, and a suction tube for sucking material from the body lumen, wherein the suction tube is connected to a branch connector and one branch of the branch connector is connected to a suction source tube which is connected to a suction source and another branch of the branch connector is connected to a vent tube, and wherein the suction source tube and the vent tube pass through a double pinch valve.

### FIELD OF THE INVENTION

The present invention relates generally to systems for navigating and imaging body lumens, such as the gastrointestinal (GI) tract, such as a system and method for performing a colonoscopy with self-cleaning features that preclude the need for preparatory cleaning of the colon or assist/clean in cases of poor preparation.

### BACKGROUND OF THE INVENTION

Many imaging devices are known for producing medical images of body lumens, such as the gastrointestinal (GI) tract. For example, endoscopy is widely used for observing, photographing tissue, and taking specimens from lesions and the like. In a conventional method of examining a colon using an endoscope, for example, the endoscope is typically manually inserted into the colon. In this manual technique, patients may often complain of abdominal pain and distention because the colon is extended or excessively dilated, thereby complicating the endoscopic procedure. There may be a risk for the colon to bleed and be accidentally perforated. Insertion of an endoscope through the sigmoid colon and into the descending colon, or through the splenic flexure, the transverse colon, the hepatic flexure or parts affected by previous operations may also be accompanied with difficulty.

### SUMMARY OF THE INVENTION

The present invention seeks to provide an improved endoscopic device for performing endoscopy in a body lumen comprising an irrigation lumen (irrigation includes, inter alia, cleaning and/or washing) that provides a jet of cleaning fluid that cleans said body lumen during imaging thereof, such as the gastrointestinal (GI) tract, as is described more in detail hereinbelow. The invention includes a variety of methods/procedures, such as cleaning while imaging, cleaning during insertion of the endoscope, cleaning while withdrawing, and any combinations thereof. The invention is described hereinbelow with reference to the GI tract, but it is understood that the invention is not limited to the GI tract and may be used for other body lumens as well.

There is thus provided in accordance with an embodiment of the present invention, a method for enhancing performance of an endoscope for use with a body lumen that has not been sufficiently cleaned for viewing with the endoscope, including assembling an endoscopic system to an endoscope, the endoscopic system including an irrigation tube that provides a flow of irrigation fluid for cleaning a body lumen, and a suction tube for sucking material from the body lumen, and causing fluid to exit the irrigation tube into the body lumen to clear debris away from a path of the endoscope so that the endoscope can view the exposed body lumen, and draining at least a portion of the debris through the suction tube.

The method may further include releasing a trapped vacuum in the endoscopic system by venting air into the endoscopic system.

There is also provided in accordance with an embodiment of the present invention an endoscopic system for use with an endoscope, including an irrigation tube that provides a flow of irrigation fluid for cleaning a body lumen, and a suction tube for sucking material from the body lumen, wherein the suction tube is connected to a branch connector and one branch of the branch connector is connected to a suction source tube which is connected to a suction source and another branch of the branch connector is connected to a vent tube which is connected to a check valve, and wherein the suction source tube and the vent tube pass through a double pinch valve that is operative to close the suction source tube and simultaneously, or immediately thereafter, open the vent tube.

### BRIEF DESCRIPTION OF THE DRAWINGS

The present invention will be understood and appreciated more fully from the following detailed description taken in conjunction with the drawings in which:

FIG. 1 is a simplified illustration of an endoscopic system with an irrigation and suction device mounted on an endoscope for clearing unwanted matter from a path of the endoscope in a body lumen, constructed and operative in accordance with an embodiment of the present invention;

FIGS. 2-1 and 2-2 are simplified illustrations of before and after mounting the irrigation and suction device on the endoscope, in accordance with an embodiment of the present invention;

FIGS. 3-1 and 3-2 are simplified exploded and pictorial illustrations, respectively, of an applicator for assembling a sheath on to an endoscopic device, constructed and operative in accordance with an embodiment of the present invention;

FIG. 3-3 is a simplified pictorial illustration of introducing the sheath onto the applicator, in accordance with an embodiment of the present invention;

FIG. 3-4 is a simplified pictorial illustration of the sheath in a contracted state on the applicator and an endoscopic device pulled through the applicator;

FIG. 3-5 is a simplified pictorial illustration of the endoscopic device pulled towards a hollow base of the applicator;

FIG. 3-6 is a simplified pictorial illustration of the endoscopic device having been pulled sufficiently such that the distal end of the endoscopic device is moved close to the hollow base of the applicator;

FIG. 3-7 is a simplified pictorial illustration of the endoscopic device pulled away from the applicator, wherein the sheath unfolds from the contracted state to an expanded state;

FIG. 3-8 is a simplified pictorial illustration of the shaft being removed;

FIG. 3-9 is a simplified pictorial illustration of the hollow base slid to abut against the proximal end of the endoscopic device;

FIG. 3-10 is a simplified pictorial illustration of an introducer for inserting an endoscopic device, in accordance with an embodiment of the present invention;

FIGS. 3-11, 3-12 and 3-13 are simplified illustrations of an additional applicator device to assist mounting the irrigation and suction device on the endoscope, in accordance with an embodiment of the present invention, wherein FIG. 3-11 illustrates an applicator cap, FIG. 3-12 illustrates a sleeve placed over an applicator and applicator cap, prior to assembling the sleeve on the endoscope, and FIG. 3-13 illustrates assembling the sleeve on the endoscope;

FIG. 4 is a simplified illustration of a valve system for use with the suction mode of the system, including the ability to vent air or other fluids, in accordance with an embodiment of the present invention;

FIG. 5 is a simplified illustration of a disposal bag used for dismounting the irrigation and suction device from the endoscope and disposing of the irrigation and suction device, in accordance with an embodiment of the present invention; and

FIGS. 6A-6B are simplified illustrations of a lubricating device for use with the endoscopic system, constructed and operative in accordance with an embodiment of the present invention.

### DETAILED DESCRIPTION OF EMBODIMENTS

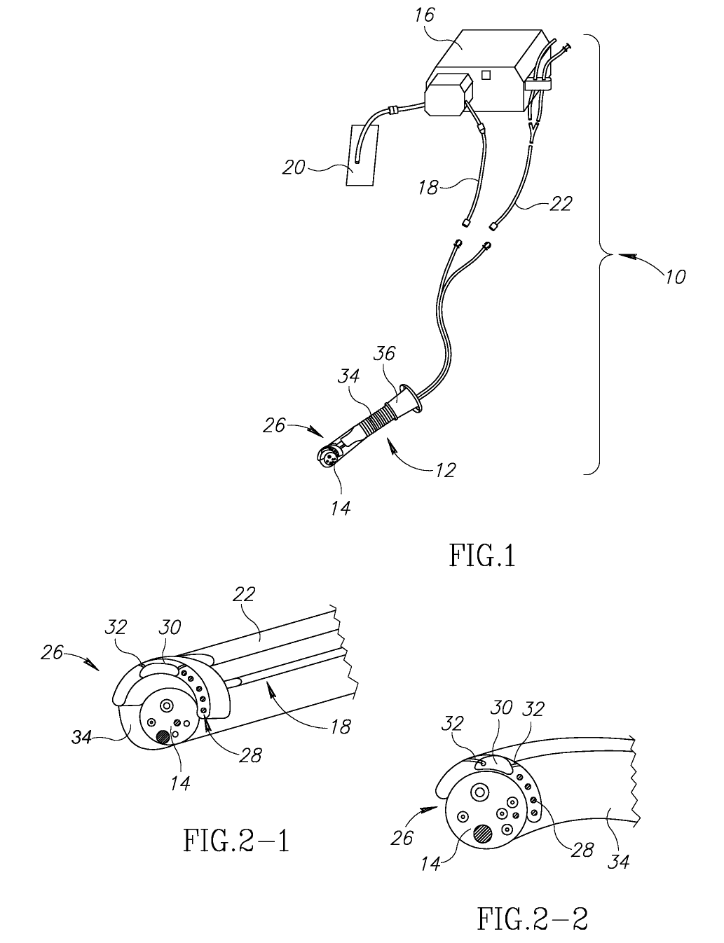

Reference is now made to FIG. 1, which illustrates an endoscopic system 10 with an irrigation and suction device 12 mounted on an endoscope 14 (such as but not limited to, a colonoscope) for clearing unwanted matter from a path of endoscope 14 in a body lumen (not shown), constructed and operative in accordance with a non-limiting embodiment of the present invention.

The endoscopic system 10 includes a controller 16 that controls operation of the system, e.g., irrigation, suction and other operational parameters. Controller 16 may be housed in a control cabinet and may include, without limitation, a power supply, processor, one or more pumps (e.g., peristaltic pumps) and one or more control valves, as is necessary for the operation desired.

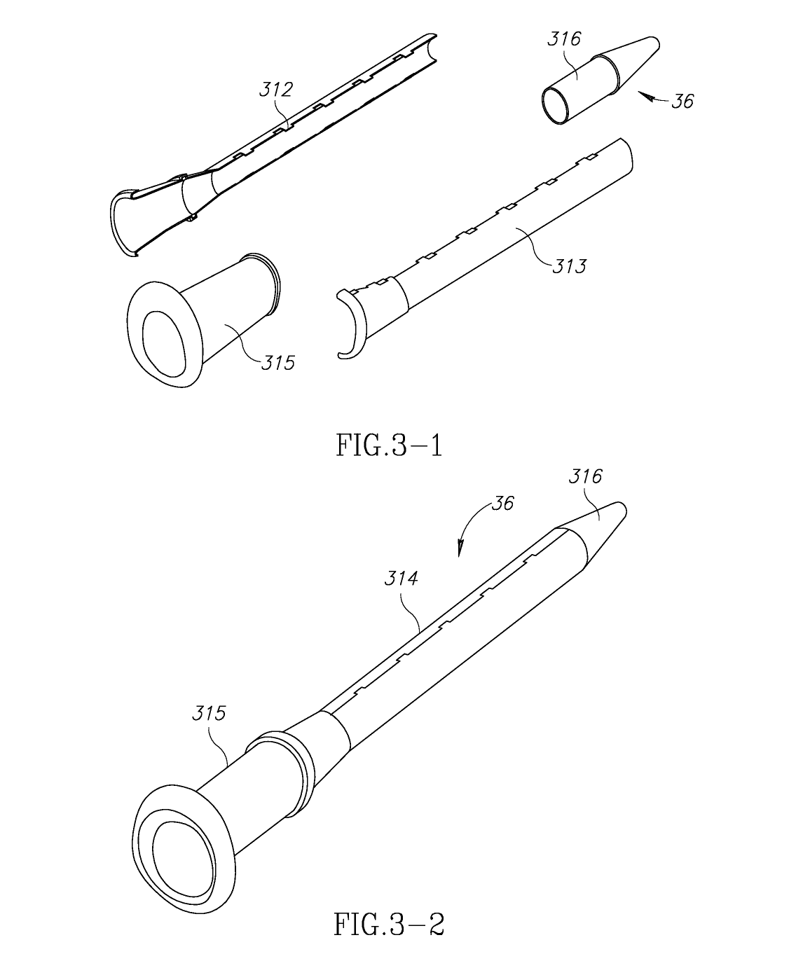

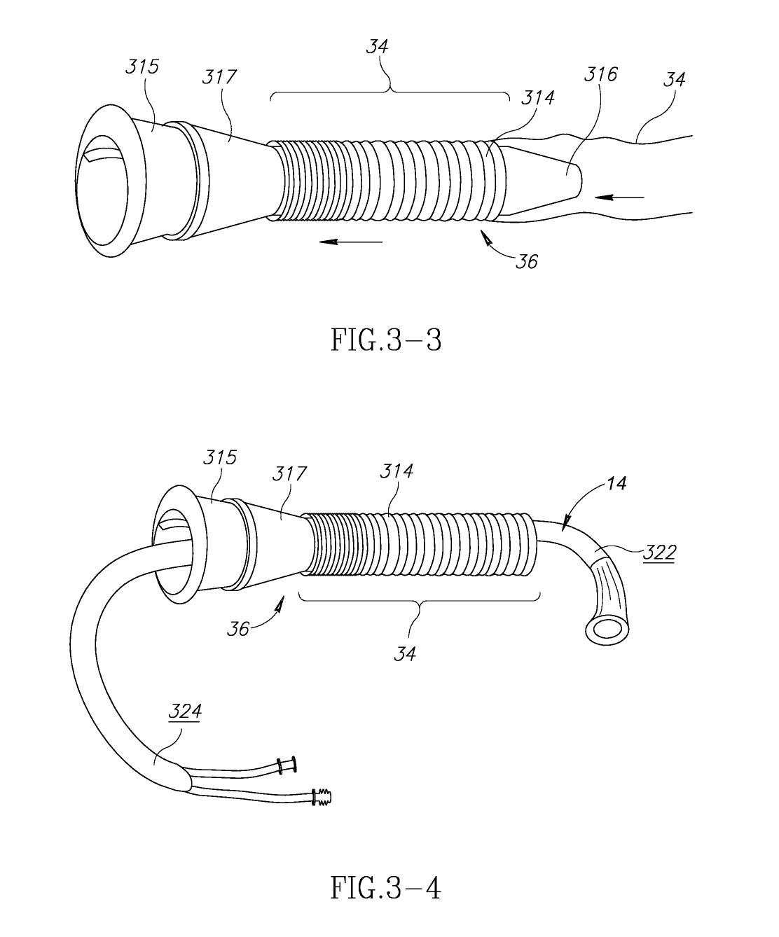

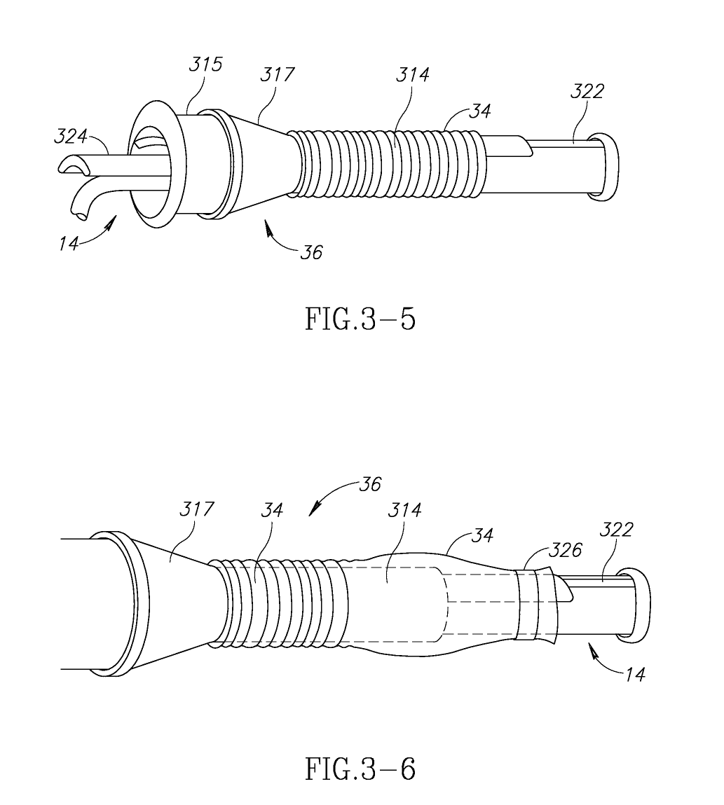

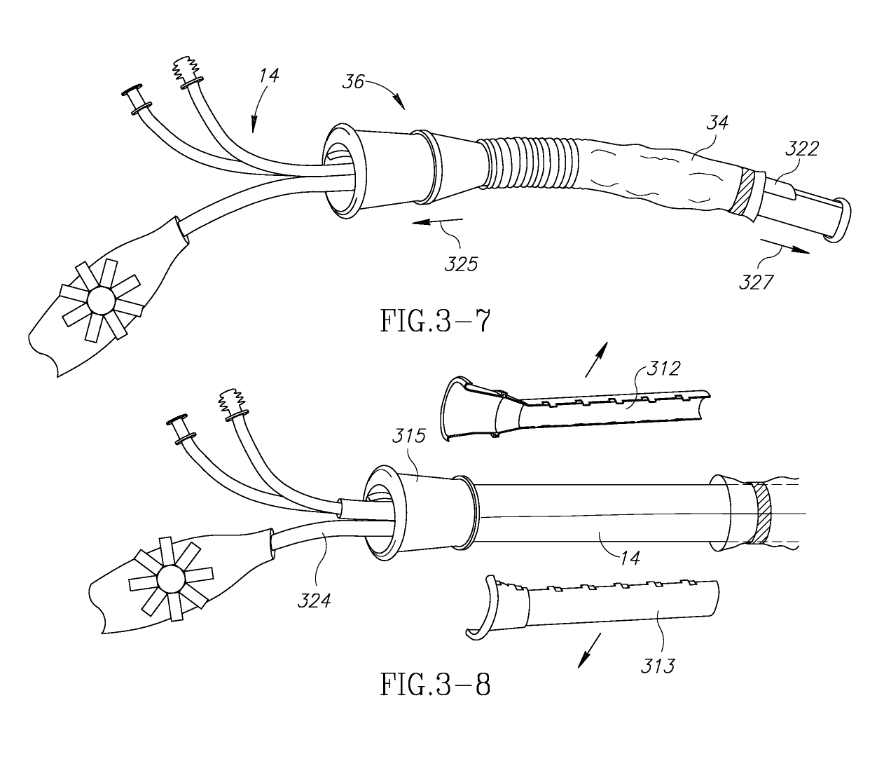

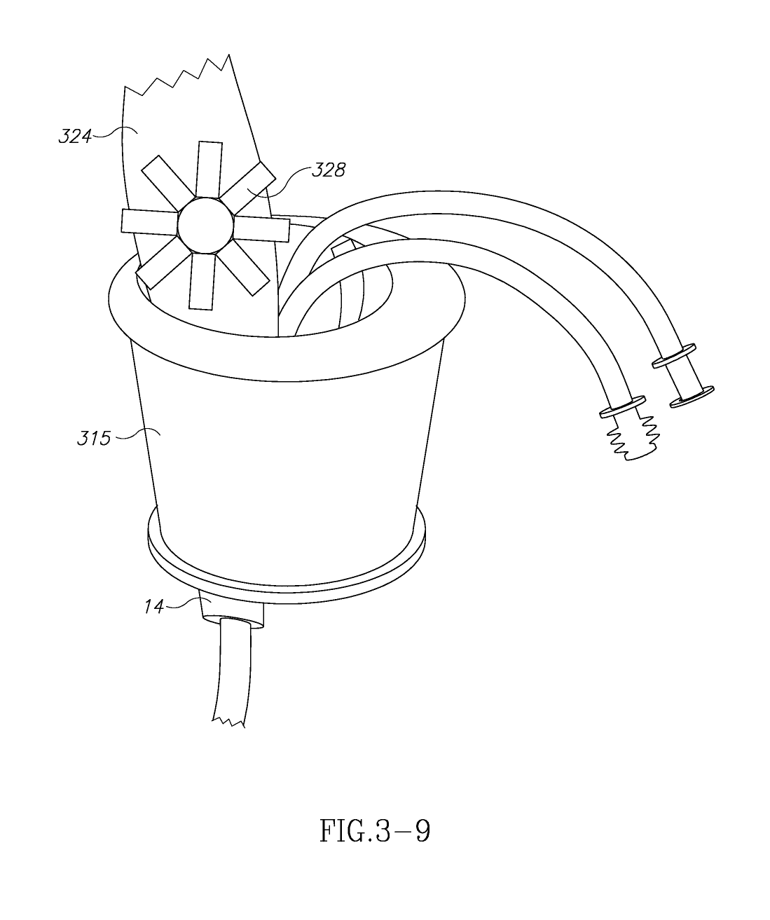

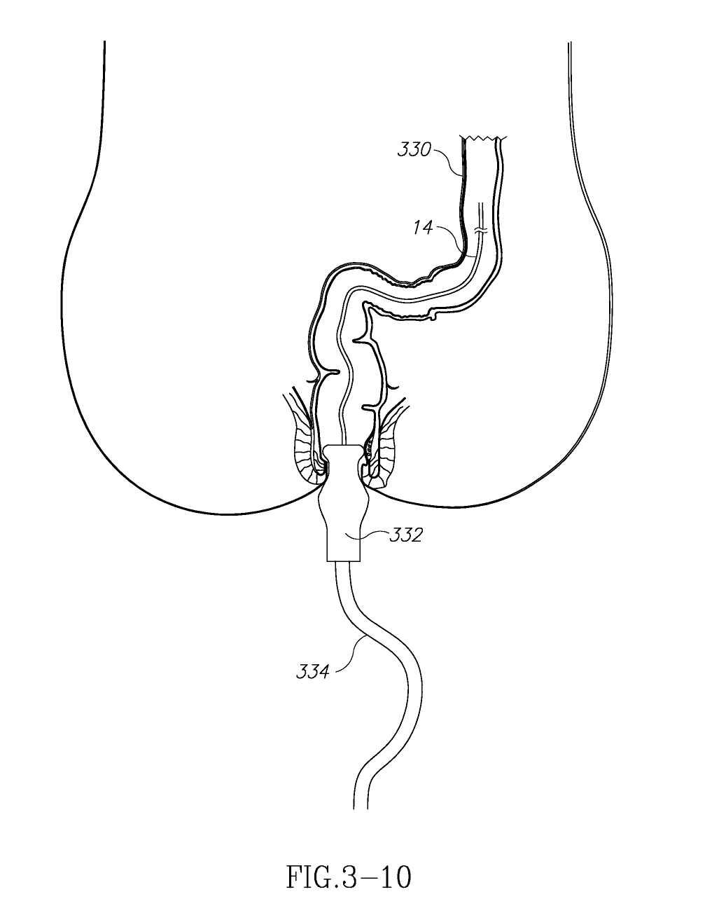

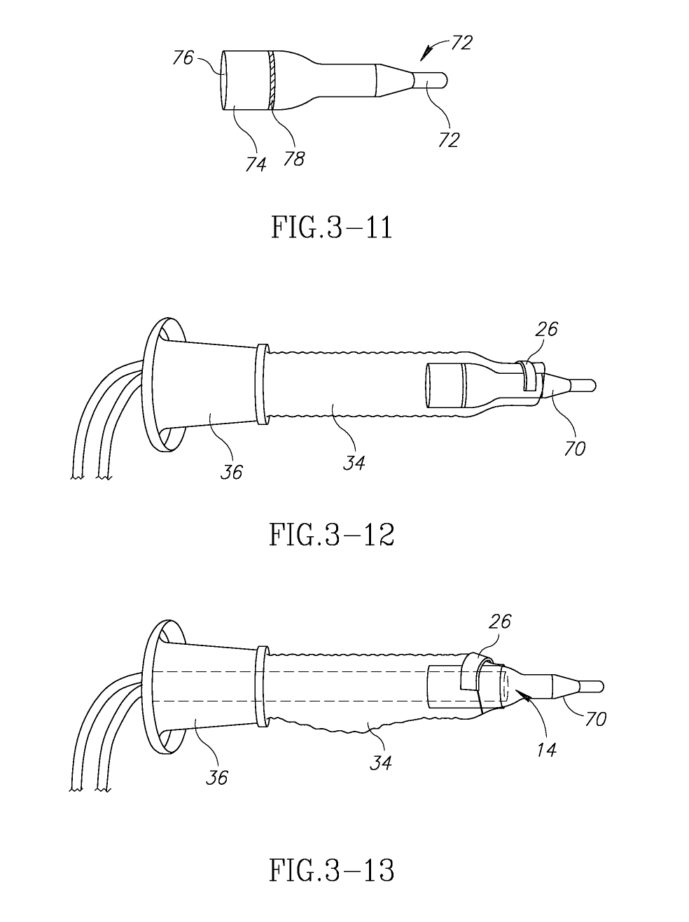

An irrigation tube 18 is connected via controller 16 to a fluid supply 20 (e.g., a water supply or other liquid or gas suitable for irrigating the particular body lumen). A suction tube 22 is connected to a suction source 24, not shown in FIG. 1, but shown in FIG. 3 and described more in detail further below. The irrigation tube 18 and the suction tube 22 connect to irrigation and suction device 12 mounted on the distal end of endoscope 14, as is now described in more detail with reference to FIGS. 2-1 and 2-2.

The irrigation and suction device 12 includes an irrigation and suction head 26, which is shaped as a partial or full ring with one or more irrigation apertures 28 and one or more suction apertures 30. In the preferred illustrated embodiment, head 26 is a partial ring with four irrigation apertures 28 and one oblong suction aperture 30. The irrigation tube 18 is in fluid communication with the irrigation apertures 28 and the suction tube 22 is in fluid communication with the suction aperture 30. One or more grooves 32 are formed adjacent suction aperture 30 to prevent tissue from getting stuck to the device during suction. The irrigation and suction head 26 is attached to a flexible sleeve 34 (made of nylon or other suitable material). FIG. 2-1 illustrates mounting the irrigation and suction head 26 on endoscope 14, prior to pulling sleeve 34 over the distal end of endoscope 14. The folds of sleeve 34 may be seen in FIG. 1, before it is pulled tautly over the endoscope 14. As seen in FIG. 1, the proximal end of sleeve 34 may be attached to an applicator 36, such as a handle or other suitable device, which assists in mounting head 26 on the endoscope 14. FIG. 2-2 illustrates the irrigation and suction head 26 fully mounted on endoscope 14, after having pulled sleeve 34 tautly over the distal end of endoscope 14. Sleeve 34 is thus used to hold the at least partial ring (irrigation and suction head 26) in place.

Reference is now made to FIGS. 3-1 and 3-2, which illustrate more in detail an example of applicator 36 for assembling the sheath on to the endoscopic device, constructed and operative in accordance with a non-limiting embodiment of the present invention.

Applicator 36 includes two shaft halves 312 and 313 (FIG. 3-1), which join together to form a cylindrical shaft 314 (FIG. 3-2). The two halves 312 and 313 may snap-fit together or may be otherwise joined, such as but not limited, by bonding, welding and the like. The two halves 312 and 313 assemble together on a (preferably cylindrical) hollow base 315, again such as but not limited, by snap-fit, bonding, welding and the like. A conical nose piece 316 fits into the assembled shaft 314. It is noted that shaft 314 has a conical portion 317 at the end that joins with hollow base 315.

Reference is now made to FIG. 3-3, which illustrates introducing sleeve or sheath 34 onto applicator 36, in accordance with an embodiment of the present invention. The sheath 34 is introduced from the end with conical nose piece 316 and abuts against conical portion 317 of shaft 314. In this manner, for example, without limitation, a 1.5 meter long sheath (also referred to as a sleeve) can be contracted (folded, bunched up) into just 15 cm. The nose piece 316 may then be removed.

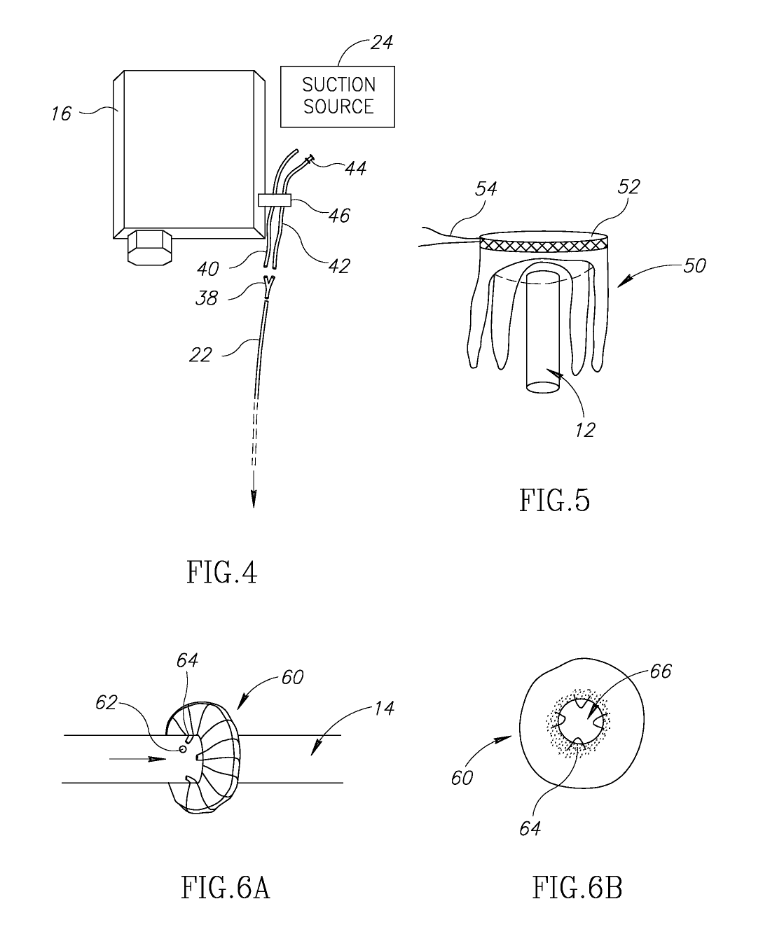

Reference is now made to FIG. 3-4. The sleeve 34 is on the applicator 36 and endoscopic device (endoscope) 14 is pulled through the applicator 36. The distal end 322 of endoscopic device 14 protrudes out of the end of shaft 314 opposite to hollow base 315 and the proximal end 324 of endoscopic device 14 protrudes out of the hollow base 315.

In FIG. 3-5, the endoscopic device 14 is pulled towards hollow base 315. In FIG. 3-6, the endoscopic device 14 has been pulled sufficiently such that the distal end 322 of endoscopic device 14 is close to the end of shaft 314 opposite to hollow base 315. Sheath 34 is now assembled on to the distal end 322 of endoscopic device 14 and secured thereto, such as but not limited to, with a mechanical fastener 326 (e.g., a band, retaining ring, clamp, etc.) or snugly tucked into a portion of the endoscopic device 14 or by any other suitable means of joining.

In FIG. 3-7, the distal end 322 of endoscopic device 14 is pulled away from applicator 36 and the sheath 34 unfolds from the contracted state to an expanded state. Either applicator 36 is pulled proximally away from endoscopic device 14 in the direction indicated by arrow 325, or endoscopic device 14 is pulled distally off applicator 36 in the direction indicated by arrow 327, or both.

Reference is now made to FIG. 3-8. After expanding and unfolding sheath 34 to the desired length, shaft 314 is removed by taking off shaft halves 312 and 313.

Reference is now made to FIG. 3-9. The hollow base 315 may be slid to abut against the proximal end 324 of endoscopic device 14, such as near a proximal handle 328 of endoscopic device 14. The hollow base 315 may serve as an introducer (like a funnel introducer) for introducing catheters, cannulas, instruments and any other medical devices into a body lumen, which are passed through the sheath 34. The sheath 34 may thus protect the patient from contamination.

After use of endoscopic device 14, hollow base 315 and sheath 34 may be discarded. The sheath 34 can be removed from the endoscopic device 14 by inverting backwards over the endoscope head, thereby preventing the endoscopic device 14 from contacting any contaminants.

Reference is now made to FIG. 3-10, which illustrates an introducer for inserting an endoscopic device, in accordance with an embodiment of the present invention.

The endoscopic device 14 (such as the device of PCT patent application PCT/US09/44595, the disclosure of which is incorporated herein by reference) is shown inserted into a body lumen 330 (e.g., the rectum and the GI tract). An introducer 332 (FIG. 3-1) may help insert the endoscopic device 14 in the rectum. Introducer 332 helps seal the opening of the body lumen (in this case the rectum), and can be used to introduce catheters, cannulas, instruments and any other medical devices into the body lumen, as well as fluids such as through a tube 334 (connected to a fluid source, not shown, such as water for cleaning the body lumen). By sealing the body lumen, introducer 332 can facilitate introducing the cleaning fluid into the body lumen and providing a cleaner environment for performing endoscopic procedures, such as those described in PCT patent application PCT/US09/4459.

Reference is now made to FIGS. 3-11, 3-12 and 3-13, which illustrate an additional applicator device to assist mounting head 26 on endoscope 14.

FIG. 3-11 illustrates an applicator cap 70, having a distal nose 72 formed with one or more tapered sections, and a proximal body 74 with a hollow end 76. A mark 78 is formed on proximal body 74 which denotes the final position of the distal end of sleeve 34 after pulling sleeve 34 over the distal end of endoscope 14 (not shown in FIG. 3-11).

FIG. 3-12 illustrates sleeve 34 placed over applicator 36 and applicator cap 70, prior to assembling sleeve 34 on the endoscope 14. The irrigation and suction head 26 is at the distal end of applicator cap 70. The distal end of sleeve 34 is placed over applicator cap 70 and applicator 36 is at the proximal end of sleeve 34.

In FIG. 3-13, the endoscope 14 is placed through applicator 36, goes through sleeve 34 and passes through the hollow end 76 of applicator cap 70. The endoscope 14 pushes the applicator cap 70 with respect to sleeve 34 until the distal end of sleeve 34 reaches mark 78. The user then simply pulls applicator cap 70 distally off the assembly and then pulls sleeve 34 proximally and tautly over the distal end of endoscope 14, thereby mounting irrigation and suction head 26 on endoscope 14 as described above.

Reference is now made to FIG. 4, which illustrates a valve system for use with the suction mode of the system, with the ability to vent air or other fluids, in accordance with a non-limiting embodiment of the present invention.

Suction tube 22 is connected to a branch connector 38 (e.g., Y-connector). One branch of connector 38 is connected a suction source tube 40 which is connected to suction source 24. The other branch of connector 38 is connected to a vent tube 42 which is connected to a check (or one-way) valve 44. Both suction source tube 40 and vent tube 42 pass through a double pinch valve 46. Double pinch valve 46 can be used to close suction source tube 40 and simultaneously, or immediately thereafter, open vent tube 42. This action releases any trapped vacuum that may exist in the system. The open vent tube 42 allows ambient air or pressurized external air (from some pressurized air source, not shown) to enter the system and flow to irrigation and suction head 26 (FIGS. 2-1 and 2-2) in order to expel or purge any debris that may block the apertures of head 26. Check valve 44 prevents any irrigation liquid or other fluids from leaking out, but at the same time allows venting air (or other venting fluid) to enter the system.

Reference is now made to FIG. 5, which illustrates a disposal bag 50, made of plastic or any suitable flexible material, used for dismounting the irrigation and suction device 12 from the endoscope 14, in accordance with an embodiment of the present invention. Disposal bag 50 is initially folded over itself many times, so that it can easily be placed over irrigation and suction device 12. Disposal bag 50 is placed over the distal end of irrigation and suction device 12 and pulled taut (thereby unfolding the folds). Disposal bag 50 has an elastic opening 52 which clings to the outer periphery of irrigation and suction device 12. As bag 50 is pulled over irrigation and suction device 12, the elastic opening 52 wipes the axial length of irrigation and suction device 12. After bag 50 is completely pulled over irrigation and suction device 12, the elastic opening 52 may be closed, such as by tying ties 54, and device 12 may be safely disposed. This ensures sterile, contactless and safe handling of device 12 by the operator.

Reference is now made to FIGS. 6A-6B, which illustrate a lubricating device 60 for use with the endoscopic system 10, constructed and operative in accordance with an embodiment of the present invention. Lubricating device 60 includes a toroidal lubricant applicator with an inner diameter to match the outer diameter of endoscope 14. Lubricating device 60 is filled with a lubricant 62 (such as, but not limited to, a water-soluble, non-fatty, antiseptic lubricating jelly and the like) which may be dispensed through nipples 64 as the device 60 is pulled over endoscope 14. The inner diameter of lubricating device 60 may be an absorbing surface 66 to help clean the outer surface of endoscope 14.

A non-limiting operation of endoscopic system 10 includes pumping a cleaning jet of fluid via irrigation tube 18 to irrigation apertures 28 for cleaning a body lumen (e.g., the gastrointestinal tract) during passage of the endoscope 14 through the body lumen, typically but not necessarily at low pressure, such as but not limited to, about 2 bar. Waste material (which may flow together with the irrigation liquid) is sucked into suction aperture 30 into suction tube 22 and exits to a waste receptacle (not shown) which is preferably disposable.

The cleaning irrigation jet can be directed in a variety of directions, such as but not limited to, distally (i.e., forward) to break up clumps and solid waste, proximally (i.e., backwards towards the anus) and to the sides, at angles, or any combination thereof. The controller 16 may be used (e.g., by means of a fluid manifold, fluid switch, electronic valve, etc.) to selectively direct the fluid flow to irrigation apertures 28, and to control the suction/vent action described above with reference to FIG. 4. The irrigation spray can be used to break up and clear away debris from the head 26 to ensure proper drainage of the fluid used to clean the body lumen. The size of the suction inlet is preferably somewhat smaller than the rest of the path through to the suction outlet so that once debris has passed the inlet it should not cause a clog with the suction lumen.

The irrigation fluid ensures that any debris, which has become stuck at the suction inlet so as to clog the inlet (because of size or shape), is cleared away from blocking the suction inlet by the force and direction of the flow. The irrigation fluid avoids sucking the body lumen wall into the suction inlet thus avoiding clogging and potential damage to the body lumen, and also preventing the sucked wall from arresting movement of the endoscope.

A variety of sequence of operations can be used in employing the endoscopic system 10, such as but not limited to, different sequences of alternating between spraying jets of fluid to clean away debris in the body lumen, sucking the debris away, cleaning debris away from the drain outlet, including different time durations of each part of the sequence, frequency and amplitude of the jet sprays and suction, all of which may be automatically controlled by the controller or manually controlled by the operator.

In another embodiment of the invention, all the above-mentioned features/channels/lumens/cleaning jets/suction and the like of the endoscopic system are incorporated within the endoscope itself, that is, supplied or built together with the endoscope by the endoscope manufacturer, or inserted through one or more of available channels within an endoscope.

The lumen for the jet flow to clean the drain may be incorporated in a working channel for passing therethrough working tools (for cutting tissue, collecting biopsy material, etc.).

The work flow may be continuous with a jet flow and suction. Alternatively, the work flow may alternate between jet flow and suction. The work flow may comprise pulses of jet flow, synchronized or not.

The jet flow may be directed proximally to create jet propulsion to propel, or assist in propelling, the endoscope distally into the GI tract. The jet flow may be continuous with suction (and may be used concurrently with the jet flow for cleaning). Alternatively, the work flow may alternate between jet flow and suction. The work flow may comprise pulses of jet flow, synchronized or not.

The suction may take place at a distal portion of the device, on the sides (via suction openings) and/or at a proximal portion of the device, or a combination thereof.

Instead of the invention being packaged as an add-on accessory, the invention may be built into the endoscope and be provided as part of the endoscope by the endoscope manufacturer.

The invention obviates the need for the aggravating pre-cleaning procedures widely used for preparing for a colonoscopy—no need for enemas, drinking awful tasting glop, diarrhea, etc. Rather the invention starts cleaning the colon upon entering the anus without causing discomfort to the patient. Imaging may be performed while passing the instrument into the colon or while extracting the instrument from the colon.

The following is a summary of a typical, but non-limiting, procedure of using the endoscopic system 10 for performing colonoscopy. First, the patient may either skip any pre-cleaning of the colon altogether or may undergo a much shorter and friendlier pre-cleaning, such as a small dosage of an enema.

A sterilized endoscope 14 is prepared by putting/attaching irrigation and suction device 12 to the distal end of endoscope 14. Alternatively, the endoscopic system 10 is pre-manufactured with the endoscope 14 as a built-in manufactured assembly, in which case the manufactured assembly comes sterilized or is sterilized prior to use.

The endoscope 14 covered with irrigation and suction device 12 is connected to the controller cabinet and the irrigation tube 18 and the suction tube 22 are connected to irrigation and suction device 12. The system is now ready to start the medical procedure.

Next, the endoscope 14 with device 12 is inserted in the colon. Fluid is introduced in irrigation tube 18 and a jet of fluid starts to clean the path for endoscope 14 in the colon. Endoscope 14 may take images of the colon as the endoscope enters the colon and/or as the endoscope exits the colon. Any waste material may be collected in a waste container. After imaging and/or other procedures performed with tools through the working channel, endoscope 14 with device 12 is removed from the colon. Disposal bag 50 is placed over the distal end of irrigation and suction device 12, pulled taut, and elastic opening 52 is closed, so that device 12 may be safely disposed. Any waste material collected during the colonoscopy is sealed in the waste container and disposed.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. A method for enhancing performance of an endoscope for use with a body lumen that has not been sufficiently cleaned for viewing with said endoscope, comprising:
fitting an endoscopic system over a distal end of an endoscope, said endoscopic system comprising an irrigation tube that provides a flow of irrigation fluid for cleaning a body lumen, and a suction tube for sucking material from the body lumen; and
causing fluid to exit said irrigation tube into the body lumen to clear debris away from a path of said endoscope so that the endoscope can view the exposed body lumen, and draining at least a portion of the debris through said suction tube.

2. The method according to claim 1, further comprising releasing a trapped vacuum in the endoscopic system by venting air into the endoscopic system.

3. The method according to claim 2, wherein venting air into the endoscopic system comprises connecting said suction tube to a branch connector, connecting one branch of said branch connector to a suction source tube which is connected to a suction source and another branch of said branch connector to a vent tube, and opening said vent tube to vent air into the endoscopic system.

4. The method according to claim 3, wherein said suction source tube and said vent tube pass through a double pinch valve, and comprising operating said double pinch valve to close said suction source tube and simultaneously open said vent tube.

5. The method according to claim 3, wherein said suction source tube and said vent tube pass through a double pinch valve, and comprising operating said double pinch valve to close said suction source tube and, immediately thereafter, open said vent tube.

6. The method according to claim 1, wherein fitting said endoscopic system over the distal end of said endoscope comprises fitting an at least partial ring over the distal end of said endoscope, said at least partial ring in fluid communication with said irrigation tube.

7. The method according to claim 6, comprising using a sleeve to hold said at least partial ring in place.

8. The method according to claim 1, comprising using an applicator to assist fitting said endoscopic system over the distal end of said endoscope.

9. An endoscopic system for use with an endoscope, comprising:
an irrigation tube that provides a flow of irrigation fluid for cleaning a body lumen, and a suction tube for sucking material from the body lumen, wherein said suction tube is connected to a branch connector and one branch of said branch connector is connected to a suction source tube which is connected to a suction source and another branch of said branch connector is connected to a vent tube, and wherein said suction source tube and said vent tube pass through a double pinch valve.

10. The endoscopic system according to claim 9, wherein said double pinch valve is operative to close said suction source tube and simultaneously open said vent tube.

11. The endoscopic system according to claim 9, wherein said double pinch valve is operative to close said suction source tube and, immediately thereafter, open said vent tube.

12. The endoscopic system according to claim 9, wherein said vent tube is connected to a check valve operative to prevent fluid leaks, but to permit venting fluid to enter the system.

13. The endoscopic system according to claim 9, further comprising an applicator having a conical portion for assembling a sheath on to the endoscope, wherein said sheath is disposed on said applicator in a contracted state, and said sheath abuts against the conical portion of said shaft.
